(19) United States
(12) Patent Application Publication
(10) Pub. No.: US 2024/0399143 A1
(43) Pub. Date: Dec. 5, 2024

(54) DEEP BRAIN STIMULATION SYSTEM

(71) Applicants: UNIVERSITE D'AIX-MARSEILLE, Marseille (FR); INSTITUT NATIONAL DE LA SANTE ET DE LA RECHERCHE MEDICALE, Paris (FR)

(72) Inventors: Emma Acerbo, Marseille (FR); Boris Botzanowski, Marseille (FR); Florian Missey, Marseille (FR); Adam Williamson, Marseille (FR)

(73) Assignees: UNIVERSITE D'AIX-MARSEILLE, Marseille (FR); INSTITUT NATIONAL DE LA SANTE ET DE LA RECHERCHE MEDICALE, Paris (FR)

(21) Appl. No.: 18/697,421

(22) PCT Filed: Oct. 14, 2022
(86) PCT No.: PCT/EP2022/078727

§ 371 Date: Mar. 29, 2024

(30) Foreign Application Priority Data
Oct. 15, 2021 (EP) 21306447.0

(51) Int. Cl. A61N 1/32; A61N 1/04; A61N 1/05; A61N 1/36

(52) U.S. Cl. CPC A61N 1/323; A61N 1/0476; A61N 1/0534; A61N 1/36034; A61N 1/36189

(57) ABSTRACT

The deep brain stimulation system has at least four stimulation pairs of electrodes. Each stimulation pair of electrodes provides an electric stimulation at a carrier frequency. The mean value between the carrier frequency of any first stimulation pair of electrodes and the carrier frequency of any second stimulation pair of electrodes defines a first mean carrier frequency. The mean value between the carrier frequency of any third stimulation pair of electrodes and the carrier frequency of any fourth stimulation pair of electrodes defines a second mean carrier frequency. The difference between the first and second mean carrier frequencies is equal to or greater than 200 Hz.

### TECHNICAL FIELD

The present invention relates to the technical field of deep brain stimulation. In particular, the present invention relates to a deep brain stimulation system, and a method for performing deep brain stimulation.

### PRIOR ART

Deep brain stimulation (DBS) is a technique commonly used in neuroscience, in particular to investigate neuronal networks or to treat neuropathology. The use of implantable electrodes in deep brain stimulation provides the benefit of a tightly controlled focality of stimulation at significant depths, certainly compared to other non-invasive neurostimulation methods.

One primary goal of neurostimulation is therapeutic, where stimulation is used as an intervention in disease. However, a second and equally important goal is neuroscientific, where stimulation is used to gain an improved understanding of neural circuits, for example how the brain controls emotional, cognitive, and behavioral responses. Meeting these two goals necessarily involves using methods of neuromodulation which allow researchers and clinicians to precisely control and evaluate the induced changes in neural activity. The advantage of animal models allows the use of invasive stimulation methods which emphasize precise control offering high spatial focality and often cell-type-specific control, methods which include pharmacological interventions, reversible cooling deactivation, faradaic stimulation with implanted microelectrodes, and most obviously optogenetics.

While being interesting and highly effective, the majority of invasive methods are impossible to translate to healthy humans or patients. Although extremely effective, opening new understanding in brain circuits and therapy, invasive electrode implantation in brain structures can induce significant tissue damage and the surgery itself due to the level of invasiveness possesses inherent risk. As a consequence, the majority of studies in humans require the use of entirely non-invasive brain stimulation (NIBS) methods.

An example of an invasive DBS stimulation for both investigative and therapeutic purposes is the use of stereoencephalography (SEEG) in epileptology. SEEG is an invasive technique consisting of the implantation of numerous intracerebral electrodes which allow the precise definition of the epileptogenic zone (EZ) in patients. From the implanted SEEG electrodes at various depths in numerous brain structures, stimulation is used to map and define the EZ and the specific tissue region to be surgically resected to achieve seizure-freedom. Unfortunately, respective surgery fails 40% of the time, illustrating the need to improve techniques for exploring brain tissue and identifying the EZ in clinical research. Certainly, the limitation of SEEG is the implantation itself, as electrodes cannot be implanted arbitrarily in brain tissue, especially not in eloquent regions or places of high vessel density such as the insula.

Initial results of NIBS utilized currents with intensities of approximately 20 mA, applied to the scalp of humans to activate areas of cortex and change behavioral responses corresponding to the activated brain regions. Although a significant achievement to stimulate cortical brain regions without implanting electrodes in the tissue, currents with such high amplitudes are extremely physically uncomfortable. From this initial study, two NIBS methods have been developed and are in regular clinical use, namely transcranial magnetic stimulation (TMS) and transcranial electrical stimulation (tES), the latter method using currents with intensities of approximately 1 to 2 mA, causing no physical discomfort. Although limitations of focality for both TMS and tES are discussed in literature, both methods have shown clear effectiveness in stimulating regions of cortex. However, one limitation of these techniques is depth, because they cannot stimulate regions below the cortex. The depth profile of stimulation is limited by the intensity of the applied stimulation and the frequency as a function of attenuation by the tissue.

Numerous non-invasive neurostimulation techniques are currently used to explore brain tissue, amongst them, the method of temporal interference (TI) shows an interesting ability to stimulate at points located away from the electrode surface. Two electric fields at two different high frequencies are applied causing an envelope frequency for stimulation at a desired point in space, as described for example in international application WO 2016/057855. The value of the stimulation frequency is equal to the difference between two applied high frequencies, and the point in space is generally related to the placement of the stimulation electrodes, as described in the article of Grossman, N. et al. “Noninvasive Deep Brain Stimulation via Temporally Interfering Electric Fields”, Cell 169, 1029-1041.e16 (2017).

Such TI techniques thus use the mixing of two high frequencies to create an envelope frequency for stimulation. Known stimulation methods distribute stimulation across multiple electrodes to reduce current density, but at the loss of focality. Moreover, the location of the stimulated zone and thus the location of the stimulation electrodes are crucial, in order to avoid the risks of provoking seizures, of disrupting healthy functions and of changing brain network.

Application CN 110604868 discloses an equipment for realizing temporal interference stimulation based on time and space rotation intersection of multiple stimulation sites. The equipment comprises a plurality of electrode combinations and an elastic cloth cap serving as a carrier. The specific placing position of each electrode combination can be adjusted according to the patient head and to actual locations of focus parts, so that the intersection of the coherent regions of the different electrode combinations covers the target stimulation region, the area of the region covered by the intersection being smaller than the area of the coherent region of any one of the electrode combinations.

Application US 2019/0366088 discloses a deep brain stimulation system having at least two focuses, which comprises: a first electrode and a second electrode, used for connecting one side of a brain scalp; a third electrode and a fourth electrode, used for connecting the other side of the brain scalp; and several signal generation units used for providing different currents to each electrode, their number depending directly on the number of focuses. Said different currents are interfered at different deep brain portions to form the different focuses.

Recently, a method, named orientation-tunable TI (ot-TI), has been described in the article Missey, F and al, “Orientation of Temporal Interference for Non-invasive Deep Brain Stimulation in Epilepsy” Frontiers in Neuroscience, 15 (2021). and allows improving known TI stimulation. Specific orientations are assigned to electrode pairs with respect to the orientation of deep brain structures for the stimulation. This method allows the targeting and stimulation of the hippocampus and allows to dramatically lower the threshold to evoke seizure-like events (SLEs) by considering the symmetry of the brain structure with respect to the symmetry of the applied TI field.

However, for certain symmetries, TI is insufficient to evoke activity at deep brain structures due to increasing stimulation amplitudes unfortunately activating unintended cortical targets.

The limits of both known TI and orientation-dependent TI are related to the amplitudes of the electric field along a given trajectory from an individual pair of stimulation electrodes. Increasing the intensity from a stimulation pair increases the amplitude of the TI stimulation envelope, however it correspondingly decreases focality by increasing the size of the stimulated region. Regions with lower thresholds to be activated along a given trajectory from an individual stimulation pair are activated, causing a loss of control over the depth of stimulation.

### Objective of the Invention

There is therefore a need to address, at least partially, the above-mentioned problems in order to provide a reliable, safe and efficient deep brain stimulation system.

### SUMMARY OF THE INVENTION

The invention notably seeks to meet this objective and its subject, in one of its aspects, is a deep brain stimulation system comprising at least four stimulation pairs of electrodes, each stimulation pair of electrodes providing an electric stimulation at a carrier frequency,
the mean value between the carrier frequency of any first stimulation pair of electrodes and the carrier frequency of any second stimulation pair of electrodes defining a first mean carrier frequency, the mean value between the carrier frequency of any third stimulation pair of electrodes and the carrier frequency of any fourth stimulation pair of electrodes defining a second mean carrier frequency,
system wherein the difference between said first and second mean carrier frequencies is equal to or greater than 200 Hz.

By increasing the number of stimulation pairs and calculating their ideal frequencies, it is possible to stimulate an area of the brain in a significantly more focal way. The invention also makes it possible to reduce the intensity delivered by each stimulation pair in order to further increase focality and reduce pain associated with stimulation electrodes on the scalp.

The system according to the invention allows the stimulation of deep brain structures, including all subcortical structures, with or without the implantation of stimulation electrodes, while maintaining and even increasing focality and depth of stimulation by increasing the number of interacting fields.

Thanks to the multi-frequency multi-pole TI system according to the invention, the different mixed fields created by the stimulation pairs interfere to create a focal stimulation point, while simultaneously targeting smaller points in space and reducing the necessary applied current from individual stimulation electrodes, thereby improving the known methods. The addition of multiple frequencies to create several interacting envelopes to evoke activity at depth with lower applied electric fields compared to known implantable DBS electrodes at the same location.

The use of multiple interacting high frequencies allows increasing focality at deep brain targets while simultaneously reducing the applied current from individual stimulation pairs of electrodes. The invention also allows using a lower electric field to evoke interictal-like events compared to focal deep brain stimulation from implanted stimulation electrodes.

The system according to the invention may be used in any kind of deep brain stimulations, in particular in peripheral nerves stimulation, neuromodulation and to treat neuropathologies. As a therapeutic stimulation method, the invention can take the known targets of stimulation and simultaneously provide a non-invasive stimulation while maintaining excellent focality, for example used in the context of epilepsy. As a neuroscientific stimulation method, the invention can provide a non-invasive method to reach subcortical areas in healthy humans and patients, providing stimulation in regions which were previously occluded from study due to the limitations of implantation in the known methods.

In the present disclosure, “carrier frequency” has to be understood as the stimulation frequency of each stimulation pair of electrodes providing the electric stimulation.

Each stimulation pair of electrodes advantageously comprises one electrode and its return electrode, creating a stimulation pair.

The system according to the invention may be configured so that the amplitude of the current passing through each stimulation pair of electrodes is chosen as a function of a predefined focal point. The envelope amplitude at the focus point, also called the hotspot, is directly dependent of the current values delivered by the stimulation electrodes. It can be noted that the more pairs, the less current delivered by each pair for the same local effect at the hotspot. The amplitude of the stimulation is thus decreased, the reduction in necessary applied stimulation being advantageously the number of pairs divided by two.

The system may comprise between four and a number n of stimulation pairs of electrodes, the difference between the carrier frequency of any first stimulation pair of electrodes and the carrier frequency of any second stimulation pair of electrodes defining a first envelope frequency, the difference between the carrier frequency of any third stimulation pair of electrodes and the carrier frequency of any fourth stimulation pair of electrodes defining a second envelope frequency, the difference between the carrier frequency of any subsequent stimulation pair of electrodes and the carrier frequency of another subsequent stimulation pair of electrodes defining a subsequent n^{th} envelope frequency, and so on, the values of the first, second, and any subsequent n^{th} envelope frequencies are equal, being especially less than 500 Hz, and being especially equal to between 180 Hz and 1 Hz.

Identical envelopes are thus created and are summed to create a “super-zone” of overlapping envelopes, namely the focal point for the stimulation. Said focal point may be the smallest relevant structure in the brain to be stimulated, for example a nucleus.

The carrier frequencies of the stimulation pairs of electrodes are advantageously greater than 1000 Hz.

The difference between any two mean carrier frequencies may be equal to or greater than 800 Hz.

The carrier frequencies are chosen so as to create a gap which does not stimulate or evoke activity.

In a preferred embodiment, the carrier frequency of a first stimulation pair of electrodes is 1250 Hz, the carrier frequency of a second stimulation pair of electrodes is 1300 Hz, the carrier frequency of a third stimulation pair of electrodes is 2150 Hz, and the carrier frequency of a fourth stimulation pair of electrodes is 2200 Hz.

The system according to the invention is advantageously configured so that the phase of the envelopes is controlled, especially by a predefined modulation which allows obtaining the desired phase. Indeed, as multiple interacting envelopes are created, the envelopes need to be in phase.

The stimulation amplitude of the electrodes may be comprised between 10 μA and 2500 μA, better between 1 mA and 2 mA.

The coordinates of the electrodes on the scalp may be calculated based at least on a predefined simulation for the stimulation, especially using the finite element method, able to predict current propagation using a predefined referential, for example a human head or a human body.

### Method

The invention also relates to a method for performing deep brain stimulation, using the system according to the invention.

The phase of the envelopes is advantageously controlled, especially by a predefined modulation which allows obtaining the desired phase.

The stimulation amplitude of the electrodes may be increased in steps of between 30 μA to 100 μA, especially in 50 μA steps. Such steps may be used for evoking seizures in a clinical environment, especially in the known Kindling protocol.

The stimulation may utilize waveforms, advantageously biphasic, especially with bipolar waveforms.

The features described above for the system apply to the method, and vice and versa.

### BRIEF DESCRIPTION OF THE FIGURES

Other features and advantages of the invention will become more apparent on reading the following detailed description and on studying the attached drawing in which:

FIG. 1A and FIG. 1B schematically illustrate an example of a system according to the invention;

FIG. 2 shows wave signals associated with the system of FIG. 1;

FIG. 3 illustrates an experimental setup for a system according to the invention;

FIG. 4 shows the creation of envelopes in known systems and in a system according to the invention;

FIG. 5 shows a comparison between known systems and a system according to the invention, regarding the created focal zones;

FIG. 6 shows a comparison between known systems and systems according to the invention, regarding the amplitudes of the signal;

FIG. 7 shows different phase modulations for obtaining desired envelope phases in a system according to the invention;

FIG. 8 shows different sizes of focal points;

FIG. 9 illustrates the principle of the deep brain stimulation system according to the invention;

FIG. 10 illustrates the repartition of subcomponent frequencies in the skull, according to the invention;

FIG. 11 shows that a multipolar temporal interference has a better efficient impact on the spiking generation, according to the invention; and

FIG. 12 illustrates that the spiking activity has been elicit in an awake monkey according to the invention.

### DETAILED DESCRIPTION

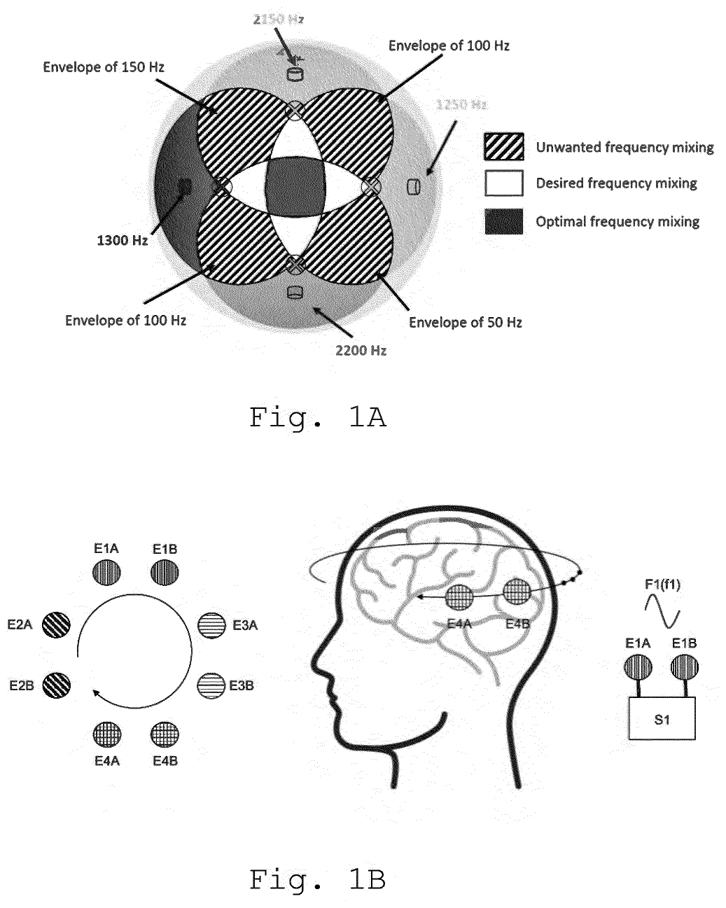

In the embodiment illustrated in FIG. 1A, a deep brain stimulation system according to the invention comprises stimulation pairs of electrodes placed on different sides of the brain scalp, each electrode having a stimulation frequency. It is to be noted that, in this figure, the electrodes are schematized either by cylinders or circles containing a cross. The difference between the mean of the two stimulation frequencies of a first stimulation pair of electrodes and the mean of the two stimulation frequencies of a second stimulation pair of electrodes among all stimulation pairs of the system is greater than 200 Hz, so that a single focal point is created inside the brain for the stimulation. Each stimulation pair comprises one electrode and its associated return electrode.

In the considered example, between two stimulation pairs of electrodes, the difference in frequency between each electrode is the same and is equal to 50 Hz. Moreover, the difference between the mean of the two stimulation frequencies of a first stimulation pair of electrodes and the mean of the two stimulation frequencies of a second stimulation pair of electrodes is even greater than 800 Hz: the stimulation frequencies of the first stimulation pair of electrodes are 1150 Hz and 1200 Hz respectively for each electrode, and the stimulation frequencies of the second stimulation pair of electrodes are 2150 Hz and 2200 Hz respectively for each electrode.

The electrodes should not act as capacitors during stimulation in order to avoid non-faradaic processes. Typical ECG electrodes, which are composed of Ag/AgCl or electrodes coated with Poly(3,4-ethylenedioxythiophene)(PEDOT)-Poly(styrene sulfonate)(PSS) may be used.

The deep brain stimulation system is also illustrated in FIG. 1B. As appearing on that figure, the system implements electrodes E in pairs, which are referenced E1A/E1B, E2A/E2B, E3A/E3B and E4A/E4B. There are at least 4 electrode pairs. These electrodes pairs are positioned on the surface of the scalp, around the head of a mammal, for instance a human individual. The position of the electrodes of a pair of electrodes may be close one to another, even at an immediate vicinity, or at various positions, for instance, at opposite positions around the patient's head. However, the positioning of the electrodes should be calculated beforehand in such a way as to have a maximum modulation at the point of interest within the brain, while minimizing the electric current delivered by each electrode. If the situation allows it, positioning electrodes on various axes, such as the axes anterior-posterior, medio-lateral or dorso-ventral, should result in a greater focal gain.

Any signal generator may be implemented in the system according to the invention, as long as it can generate high frequencies as disclosed herein, and it is possible to modify the phase of the signal. Generally, the generator is connected to one or more electric current sources. The current sources themselves electrically connected to the electrodes. Each pair of electrodes is connected to an individual signal source and the highest stimulation intensity is then concentrated between the electrodes of each such pair. As shown in FIG. 1B, each pair of electrodes is connected to a current source S1 for the first pair of electrodes E1A/E1B, S2 for the second pair of electrodes E2A/E2B, S3 for the third pair of electrodes and S4 for the fourth pair of electrodes. Only the current source S1 is illustrated in FIG. 1B. The current sources are independent. The current source S1 provides a signal function F1 from pair E1. The current source S2 provides a signal source F2 from pair E2. The current source S3 provides a signal function F2 from pair E3 and the current source S4 provide a signal function from pair E4. The signal functions are with corresponding frequencies f1, f2, f3 and f4. The signal function F can be any periodic signal such as sine wave, square wave, monopolar or bipolar pulses, with the frequency f (f1 for F1, f2 for F2, f3 for F3 and f4 for F4.

As can be seen in FIG. 1A, the stimulation pairs create envelopes in time, each at the difference in frequency. The hatched parts correspond to the unwanted frequency mixing, the white parts correspond to the desired frequency mixing while the dark part corresponds to the optimal frequency mixing, leading to a single focal point according to the invention.

The wave signals of FIG. 2 show the interaction between the envelopes. These signals are all sine shaped, because such signals make it easier to demonstrate the effect of interacting fields.

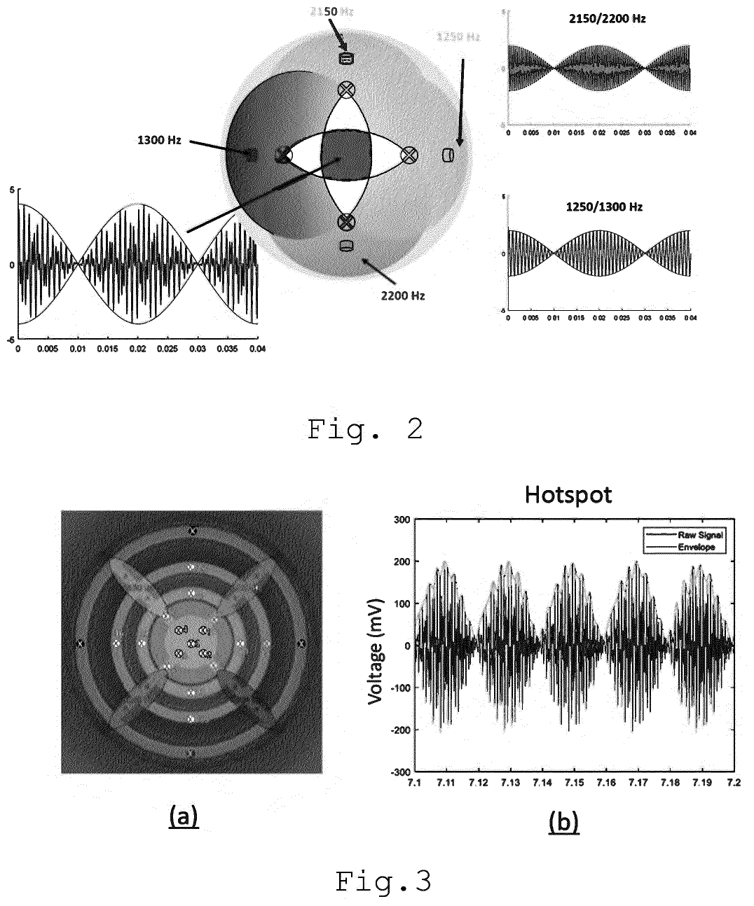

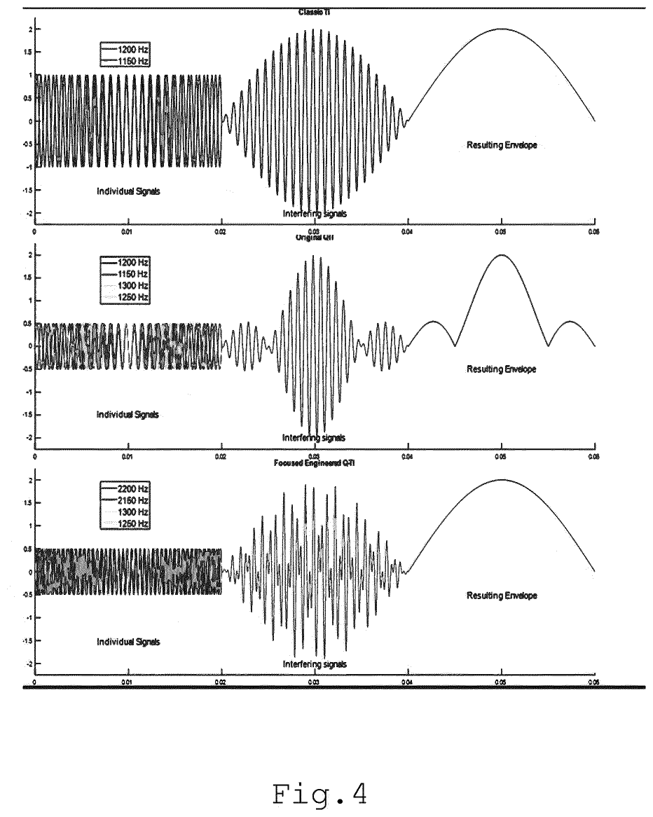

FIG. 3 illustrates an experimental setup for a stimulation system according to the invention. The system used for the stimulation comprises two stimulation pairs of electrodes, the stimulation frequencies of the first stimulation pair of electrodes are 1150 Hz and 1200 Hz respectively for each electrode, and the stimulation frequencies of the second stimulation pair of electrodes are 2150 Hz and 2200 Hz respectively for each electrode. Increasing the number of electrodes allows to reduce localized current density for the same amplitude at the hotspot, in the center. Moreover, the difference between the frequencies at each stimulation pair of electrodes allows removing rogue frequencies at the hotspot and the rest of the medium.

FIG. 4 shows the creation of envelopes by signals interfering constructively and destructively. The first diagram from the top corresponds to a system with only two stimulation pairs of electrodes, with carrier frequencies equal to 1200 Hz and 1150 Hz. The second diagram corresponds to a system with four stimulation pairs, with carrier frequencies equal to 1200 Hz, 1150 Hz, 1300 Hz and 1250 Hz. The third diagram corresponds to a system according to the invention, with four stimulation pairs of electrodes, with carrier frequencies equal to 2200 Hz, 2150 HZ, 1300 Hz and 1250 Hz. The amplitude of the envelope remains the same when the amplitude delivered by the pairs is decreased thanks to the increase in the number of pairs.

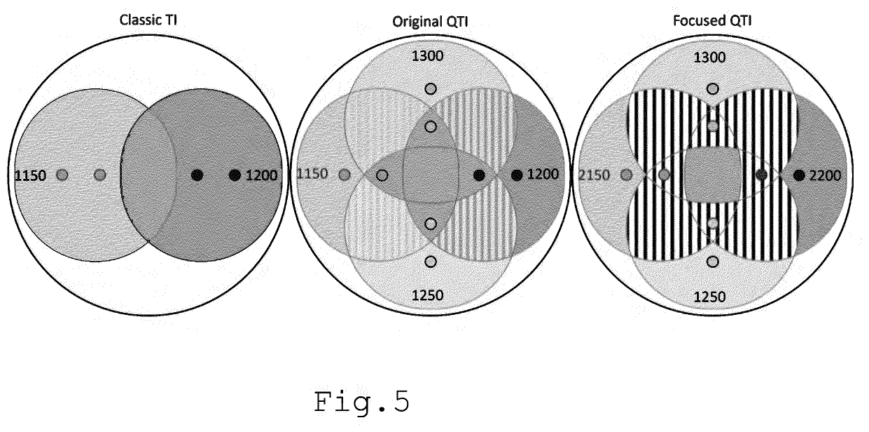

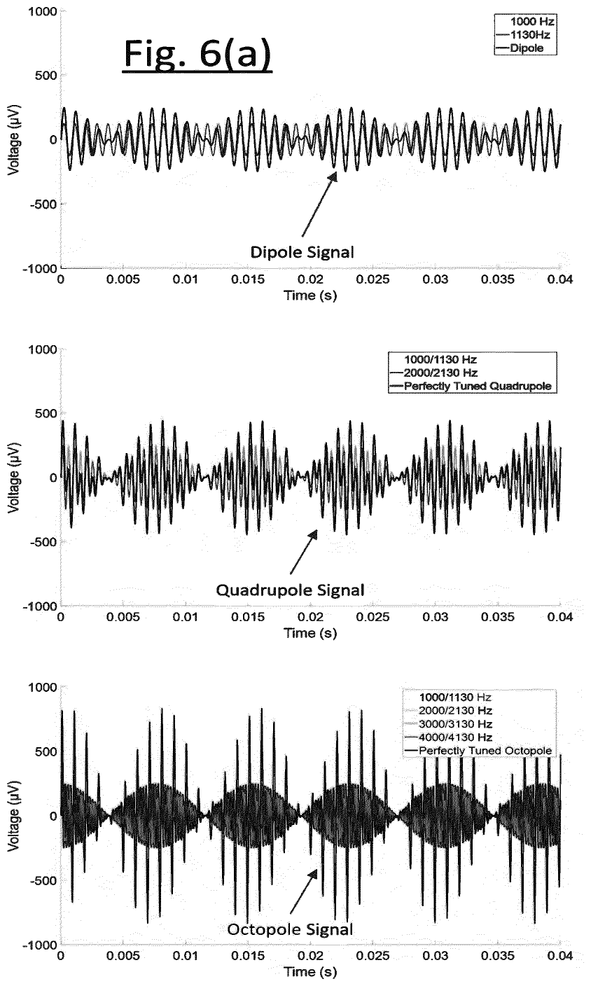

FIG. 5 shows envelopes created in time by the stimulation pairs of electrodes. The left diagram corresponds to a system with only two stimulation pairs of electrodes, with carrier frequencies equal to 1200 Hz and 1150 HZ, the one in the middle corresponds to a system with four stimulation pairs of electrodes, with carrier frequencies equal to 1200 Hz, 1150 Hz, 1300 Hz and 1250 Hz; and the one on the right corresponds to a system according to the invention, with four stimulation pairs of electrodes, with carrier frequencies equal to 2200 Hz, 2150 Hz, 1300 Hz and 1250 Hz.

The hatched parts correspond to the unwanted frequency mixing, the white parts correspond to the desired frequency mixing while the grey parts in the middle correspond to the optimal frequency mixing. In the system according to the invention, this leads to a single focal point, as can be seen in the diagram on the right. One can mark that the middle grey parts in the known systems are much bigger than in the one according to the invention.

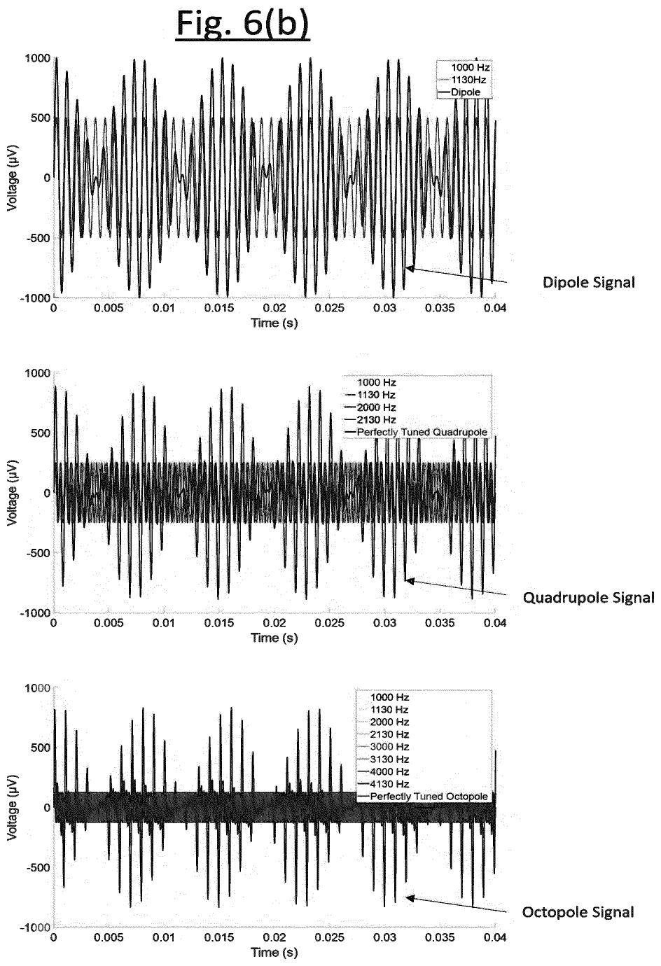

FIG. 6 shows different amplitudes for the current passing through each stimulation pair of electrodes, leading to a predefined focal point.

In FIGS. 6(a) and 6(b), the left diagram corresponds to a system with only two stimulation pairs of electrodes, with carrier frequencies equal to 1000 Hz and 1130 Hz, the diagram in the middle corresponds to a system with four stimulation pairs of electrodes, according to the invention, with carrier frequencies equal to 1000 Hz, 1130 Hz, 2000 Hz and 2130 Hz, and the diagram on the right corresponds to a system with eight stimulation pairs of electrodes, according to the invention, with carrier frequencies equal to 1000 Hz, 1130 HZ, 2000 Hz, 2130 Hz, 3000 Hz, 3130 Hz, 4000 Hz and 4130 Hz. One can note that the difference between the mean carrier frequency of the first and second stimulation pairs and the mean carrier frequency of the third and fourth stimulation pairs is equal to 1000 Hz, as well as the difference between the mean carrier frequency of the fifth and sixth stimulation pairs and the mean carrier frequency of the seventh and eighth stimulation pairs. On the other hand, the values of the envelope frequencies are less than 500 Hz, being equal to 130 Hz in this example. One can note that the reduction in necessary applied stimulation advantageously is the number of pairs divided by two, for example 4-pairs=½ the necessary amplitude, 8-pairs=¼ the necessary amplitude.

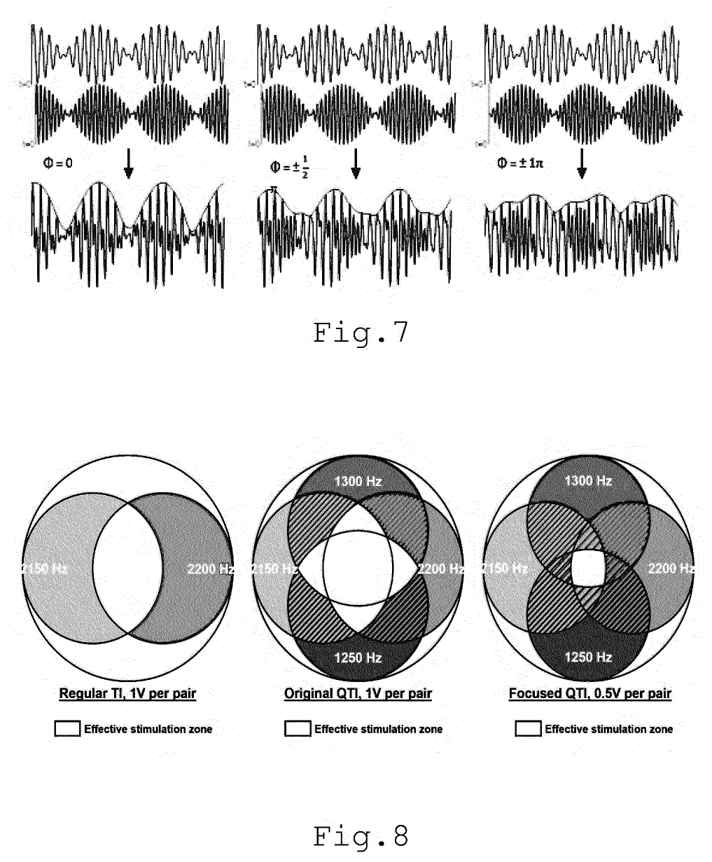

In the example shown in FIG. 7, the phase of the envelopes is controlled by using a predefined modulation which allows obtaining the desired phase for the envelopes. In the invention, as multiple interacting envelopes are created, the envelopes need to be in phase.

FIG. 8 shows different sizes of effective stimulation zones, or focal zones, for different methods: the left figure corresponds to a known TI with two stimulation pairs of electrodes, the figure in the middle corresponds to a QTI with four stimulation pairs, and the right figure corresponds to the system according to the invention with four stimulation pairs configured so that the amplitude of the current passing through each stimulation pair of electrodes is chosen as a function of a predefined focal point. The invention thus allows obtaining a better focus of the signal for the stimulation, with half the current amplitude, as can be seen in the figure.

Further experiments as described hereunder have been carried out.

### Data Collection:

In vivo, anesthetize: Data was obtained from one male rhesus macaque monkey (macaca mulatta, weighing 15 kg), as part of a planned endpoint. All surgical and experimental procedures conformed to the policies of the Canadian Council on Animal Care and the National Institutes of Health on the case and use of laboratory animals and were approved by the Animal Use Subcommittee of the University of Western Ontario Council on Animal Care. Prior to the procedure described below, the animal had a Utah array implanted in the left prefrontal cortex which had been removed 18 months earlier, as well as a pre-existing cranial implant of dental acrylic. It was relied on stereotactic coordinates (Paxinos, 2000) and anatomical MRI and CT images to plan out the placement of recording electrodes.

On the day of the procedure, the animal was induced with ketamine and medatomidine, anesthetized via propofol constant rate infusion and isoflurane inhalation, and positioned in a stereotactic frame. The acrylic implant was then removed. 2-mm diameter burr-hole craniotomies were performed at pre-determined sites to permit access of sEEG recording electrodes (Alcis Depth Coagulation Electrode). Each sEEG electrode consisted of 15 recording contacts staggered over 51 mm (contact length 2 mm, inter-contact distance 3.5 mm, diameter 0.8 mm). Eight craniotomies were positioned to permit access of recording contacts within or near the right hippocampus. A ninth craniotomy was positioned to permit access to the left hippocampus. Following each of these nine craniotomies, the sEEG electrode was lowered through a guidance screw (Alcis 2023VG, 15-25 mm length) that was secured to the skull via dental acrylic. All recording contacts were connected to an Intan recording system.

Following insertion of the sEEG electrodes, 16 locations on the scalp were selected for placement of TI stimulation electrodes. Stimulation locations were placed on that portion of the scalp that ringed the now-removed acrylic implant. The scalp was shaved and cleaned with rubbing alcohol. Stimulation was delivered through standard ECG monitoring electrodes (Medi-Trace 230); portions of the adhesive part of each electrode were trimmed in order to fit all electrodes on the scalp. Each electrode was connected to the output of a Digitimer DS-5 stimulator, which itself was connected to a waveform generator (Keysight EDU33212A). Connections were arranged to permit delivery of multi-pole TI through up to 4 pairs of stimulators.

The contact n°3 of the electrode D was chosen as our target point, thus this channel was monitored during the setting of stimulation parameters in the experiment. The amplitude of each of the 16 stimulators composing the octopole were calibrated so each would deliver a current resulting in a sine wave of around 375 μV at the target point. As the distance from the stimulators and the target was different in each case, the amplitude delivered by each stimulator was different. This was made to ensure that every envelope composing the octopole had the same amplitude. After amplitude calibration, the first stimulation pair (1975 and 2025 Hz) was powered and recorded. A second pair (2975 and 3025 Hz) was added, the phase of both signals composing the second pair was modified until a single 50 Hz envelope was obtained. This process was repeated for the third (3975 and 4025 Hz) and fourth pairs (975 and 1025 Hz) to obtain the octopole multipolar TI (mTI). The fourth pair was then turned off, and all other pairs amplitude was increased to regain initial amplitude of 50 Hz envelope to create the hexapole mTI. To create the quadrupole, third pair was turn off, and the amplitude of the first and second pair were increased to create an envelope with an amplitude similar to the initial octopole. Finally, the second pair was also turned off, and the first pair of electrodes was turned up to attain the envelope amplitude of the previous configurations.

Following completion of data collection, all sEEG electrodes were cut at the top of the guide tube, and each sEEG was glued to the guide tube using medical grade silicon. The animal was then deeply anesthetized with a bolus of propofol and high percent isoflurane and euthanized via trans cardiac perfusion with cold saline followed by 4% PFA (performaldehyde). Care was taken during euthanasia and perfusion to ensure that the guide tubes were not disturbed, and that the sEEG electrodes remained stable within the guide tube. A post-mortem CT was performed to reconstruct the location of the sEEG electrodes, via the software MRIcroGL 1.2.20210317 x86-64.

In vivo, awake: To further test the impact of the mTI stimulation, experiments on awake monkey Rhesus were performed. On these set of stimulations, the superior colliculus was the target of the different sessions of stimulation. On 2 consecutive days, a 16 channels electrode was inserted into the superior colliculus in order to record electrophysiological data and the artefact of the several TI stimulations. The frequencies used here were f1: 1950 Hz & 2050 Hz f2: 3950 Hz & 4050 HZ f3: 5950 Hz & 6050 Hz f4: 7950 Hz & 8050 Hz to get a 100 Hz envelope, currently used in superior colliculus stimulation. The position of the skin electrodes changed also to match with the new target. No behavioral tasks were performed but the position of the eyes was recorded. Stimulations sessions-dipole, quadrupole, hexapole, octupole, sham-contained: 30 TI stimulations for 200 ms on 2 minutes recordings.

### Data Analysis:

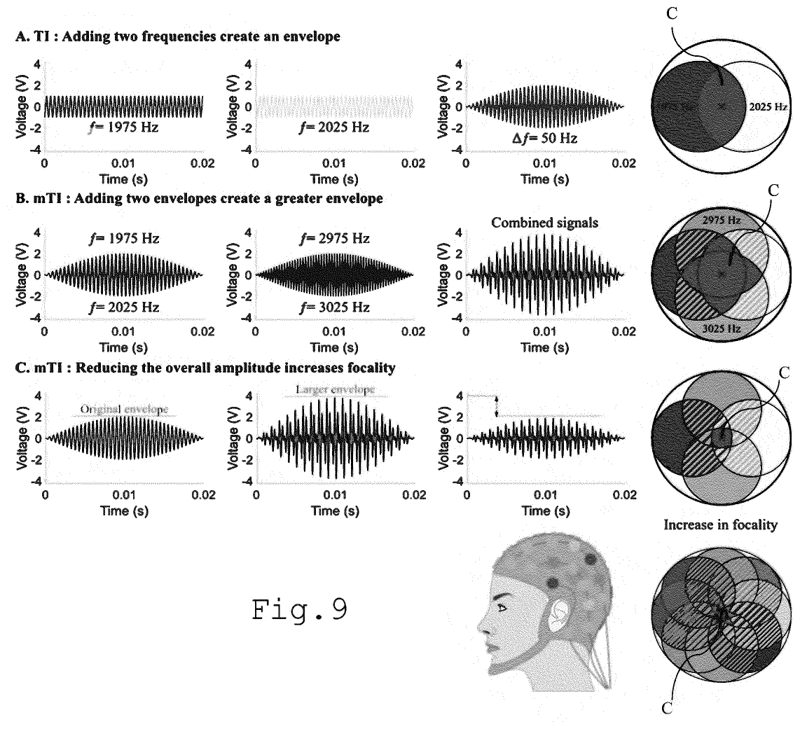

Simulations: Simulated signals were obtained by plotting simple sine functions in Matlab R2020b such as “sin(2*pi*frequency*(t))”, amplitude modulated signals were obtained by adding several sine functions. Graphical illustrations of the field distribution in FIG. 9, C, were created with the vector graphics software Inkscape 1.0.2-2.

As shown in this FIG. 9, A, on the top of the figure, illustrates an example of a set of frequency used in Temporal Interferences, wherein two sinusoidal signals of 1V and respectively of 1975 Hz and 2025 Hz interact to create a 50 Hz amplitude modulated signal. FIG. 9, B is an illustration of the addition of two 50 Hz amplitude modulated signal, wherein the first 50 Hz amplitude modulated signal is created from the interaction of a 1975 and a 2025 Hz signals, the second from a 2975 and 3025 Hz. When these two 50 Hz events interact, they create a greater 50 Hz event. FIG. 9, C is an illustration of the focality increase due to the addition of a new envelope, wherein the left panel represents the spatial distribution of a classic dipole of Temporal Interference and the signal that would be recorded at the cross. Middle panel represents the same situation with another dipole added, creating a quadrupole with its dedicated signal. When these two dipoles see their current halved, the region referenced C, where a significant interference can be observed, is drastically reduced while conserving the same amplitude, see middle and right panels.

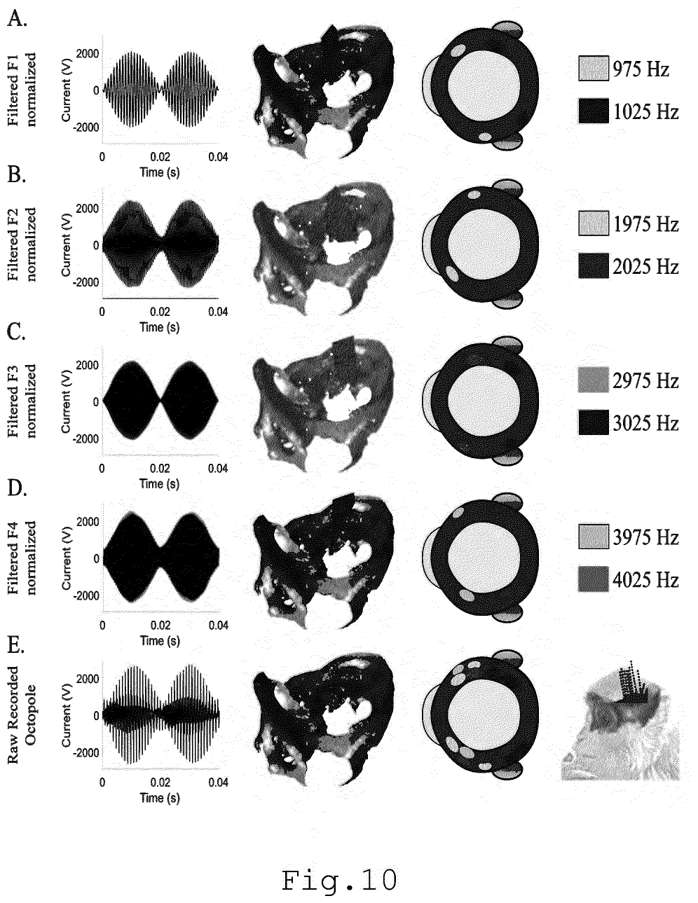

In vivo, anesthetize: The recording sample presented in FIG. 10, E was filtered with a digital filter to remove noise in order to highlight the stimulation components.

FIG. 10 as a whole illustrates the repartition of the subcomponent frequencies in the skull. On the left panels of this figure are displayed the value of the signal recorded at the point of interest. On the middle panel are displayed all the electrodes contacts which recorded at least 80% of the value of the maximum amplitude modulated signal. On the right panel, a top-down sketch of the monkey indicates the frequency and the position of the electrodes used for each configuration. The signal has been filtered to decompose the signal in multiple TI (A. for 975|1025 Hz, B. for 1975|2025 Hz, C for 2975|3025 Hz, D for 3975|4025 Hz), only E. was left unfiltered (Octopole configuration).

The signals in FIGS. 10, A, B, C and D are signals created from the original recording in FIG. 10, E, high pass and lowpass filters were used to be able to distinguish the different components composing the octopole. An envelope function was applied to the signal recordings of all the contacts with peak mode selected and a peak separation of 1.7 ms. The envelopes were segmented in samples of 20 ms each and the maximum and minimum point were used to determine the amplitude of the envelope. These values were then averaged. Raw signals and envelopes of the different pairs were then normalized so their amplitude modulated signal would match the amplitude modulated signal of the octopole at the target point, the contact n°3 of the electrode D.

The skull geometry was reconstructed from the CT scan with the software InVesalius 3.1.1 and was exported to Blender 3.1 where it has been simplified through a decimation process, the top part of the skull was also removed with Boolean operations to allow to see through. For each parameter (F1, F2, etc.) the values above their 8^{th} decile were plotted at their respective coordinates, they were then connected to create a volume. The skull was also plotted to give a spatial cue.

To detect events (spikes) in the monkey under anesthesia, a semi-automatic detection on the signal was performed. The automatic detection part was processed by using Delphos software, a detector of spikes and oscillations used mainly in clinical research. Delphos is based on a method of whitening (Z_{H}_{ₒ}) the time-frequency spectrum to optimize the signal to noise ratio at each frequency. Events of interest are detected while they are above a specific threshold in the spectrum. Before to apply Delphos on the signal, it was down sampled at 3750 Hz and cleaned to remove artefact/noise. To do so, Independent Component Analysis (ICA) available on AnyWave was used. To compute ICA, infomax method with nbcomponent=nb seeg contacts was performed. The component was labeled as bad by an expert (EA). Then the seeg signal was reconstructed without the bad component which make the signal clearer and improve delphos detections. Once events detected, they were reviewed by using AnyWave software, a visualization software for electrophysiological data. The events were accepted/rejected by the reviewer. Finally, to be comparable in between each other, a z score ((z score=x-mean(x)/sd(x)) of the spiking rate (spikes/min) has been calculated for each sessions of stimulation (dipole, mTI and dipole powered up). This allowed to distinguish which electrodes had a spiking rate higher than the average recorded by all the electrodes during each sessions. After that, a spearman correlation has been performed to see if the z score of the spiking rate (for all the recordings site in electrode D) is following the amplitude of the envelope of stimulation.

In vivo, awake: For the analysis in the awake monkey, spiking detection has been done manually for spiking activities having a frequency [130 300] Hz. As an eye movement can lead to a spike, all the stimulations with a behavioral response were removed from the analysis. To analyze the impact of the stimulation on the electrophysiological activity recorded, simulations evoking at least 1 spike on at least 1 channel were counted to test if there is an impact. To do so, a Khi 2 test was performed to know if there is at least one method of stimulation (dipole, quadrupole, hexapole, octupole) which lead to a spiking activity.

### Results:

### Multipolar Temporal Interference Principle:

The classical TI stimulation technique relies on the ability of high frequency currents to penetrate living tissue relatively unhindered compared to low frequency currents (inferior to 800 Hz) as tissue permittivity tend to increase with frequency. The TI principle is based on the interaction of two high frequency currents, superior to 1 kHz, suffering from a slight difference. This frequency difference will create a temporal interference that is both predictable and regular (with a frequency equal to the difference between the two carrier frequencies) as seen in FIG. 9.A. The neuronal or muscular tissues will only be modulated when they are stimulated by these two frequencies, namely when the tissue experiences the interference, as it is generally accepted that frequencies above 1 kHz do not stimulate tissue.

It is to be noted that all the tissue which experiences the interference can be modulated when the amplitude of the envelope exceeds a threshold. The only two pairs of electrodes used in the initial temporal interference technique offer little freedom in the spatial configuration of the stimulators, which often results in a large area of effective stimulation, especially in deep structures. In order to avoid to modulate unwanted nodes in the brains or neighboring nerves in the case of peripheral nerve stimulation, the number of stimulating pairs is increased to improve the focality of stimulation, namely the ability to modulate a targeted area without disturbing the surrounding area. This technique relies on the addition of several envelopes having the maximum amplitude of envelope at the same spot, as shown in FIG. 9.B.

With supplementary stimulators, the current provided by each stimulator can be turned down to achieve the same effect at the targeted zone as with the initial technique of Temporal Interference while offering a better focality, see FIG. 9, C. When performing this multipolar interference technique, a few things need to be considered: the frequency sets chosen to create envelopes should respect certain criteria: each envelope should be of the same frequency, the frequency pairs used to create these envelopes should be at least 1 kHz apart to prevent the creation of unwanted interferences. The phase of each envelope should be considered at the targeted zone, they should be synchronous at this point to deliver the most efficient stimulation, when asynchronous (outside the targeted zone), it will decrease the efficiency of stimulation, thus also improving the focality of stimulation. This technique works better when stimulator pairs are in different planes, allowing to really “lock” the targeted zone in 3 dimensions.

Hence, the theory for the elaboration of multipolar TI stimulation is demonstrated. It has been shown that it was possible to reach a smaller focality by adding stimulating pairs.

This was then applied on an alive animal to analyze the behavior of the various pairs of frequencies.

### Demonstration of Focality Gain Using Multipolar TI in a Macaque

As the location of each electrode pairs is different, it can be observed that the distribution of the amplitude modulated signal is different for all the dipoles, as shown in FIG. 10, A-D, while conserving the same value at the targeted contact. When all pairs are powered in an octopole configuration, as shown in FIG. 10, E, and their current is reduced to match the same amplitude of the target point, it can be observed that the volume to which the recording electrodes are subjected to at least 80% of the maximum amplitude of the envelope in the skull is greatly reduced. Thus, this configuration allows to stimulate the point of interest while reducing the neighboring volume strongly subjected to temporal interference.

By adding several pairs of electrodes, it has been shown the possibility to increase the focality of the TI stimulation and in meantime decreasing the amount of current injected for the stimulation. It was then wanted to analyze if this stimulation had an impact on the electrophysiological data in an anesthetize monkey.

### Activity Evoked by Multipolar Temporal Interference Stimulation:

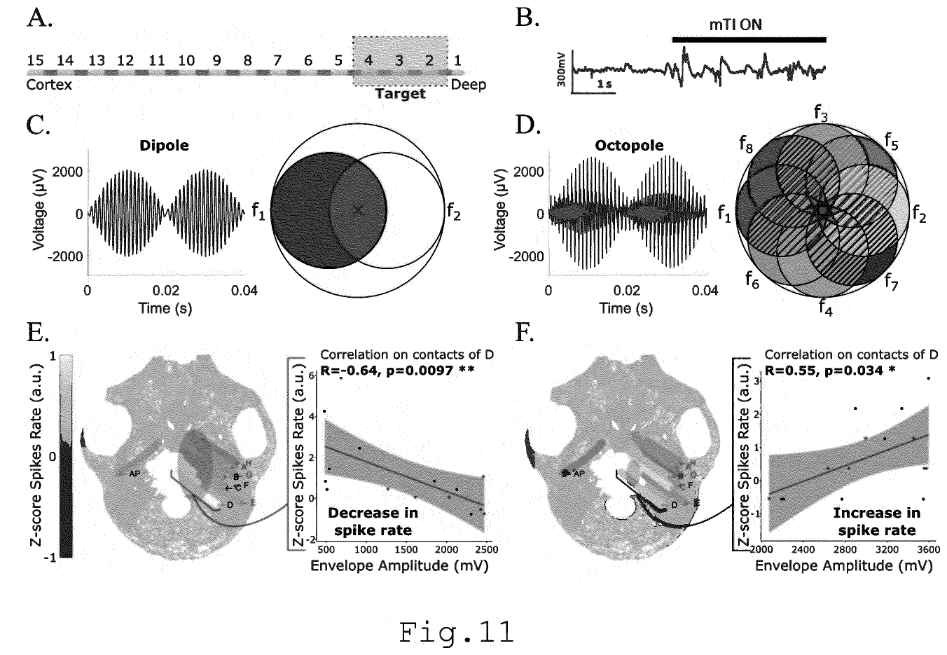

In order to determine if it is possible to evoke any electrophysiological response of the stimulation, an implantation of 9 stereoelectroencephalography (SEEG) electrodes was performed in a monkey under deep anesthesia. These SEEG electrodes have 15 recording sites along them, and are mainly used in clinics to record and stimulate patients with epilepsy 16, as shown in FIG. 11, A.

This figure illustrates that multipolar TI has a better efficient impact on the spiking generation. In FIG. 11, A 9 stereoelectroencephalography electrodes are implanted in the brain of the monkey. 8 are implanted on the right side and one in the contralateral side of the stimulation. The target chosen during the experiment was the contact n°3 of an electrode, where all the dipoles TI were synchronized. In FIG. 11, B, spiking detection has been done during the TI and multipolar TI stimulation. The frequency of 50 hz is well known to evoke spiking with implantable and dipole TI Stimulation. In FIG. 11, C, it is illustrated simulation of the envelope at the target for a dipole stimulation (classic TI), each shade of gray representing a frequency of stimulation. In FIG. 11, D, it is illustrated simulation of the envelope at the target for an octopole (8 different frequencies, multipolar TI), each shade of gray representing a frequency of stimulation. In FIG. 11, E. F., Z-score is the illustrated score (Z-score=x-mean(x)/sd(x)) of the spike rate detected for each electrode in the different conditions (Dipole and Octopole respectively) to compare the impact of the stimulation in between the stimulation sessions. A positive value describes a higher spike rate compared to the others recording electrodes. Correlation (spearman) between the spike rate detected with the amplitude of the corresponding envelope. Only the octopole are positively correlated with the induction of spikes. Even if the dipole has its amplitude of stimulation increased, it cannot induce spiking as multipolar TI-octopole.

For the experiment, the SEEG electrodes were used to record at different points in the brain. It was chosen to perform a 50 Hz stimulation with envelope, a frequency well known to evoke spiking activity in rodents 17 and use in the elaboration of a kindling model of epilepsy 18. Then, it was expected a local generation of spiking activities within this area (FIG. 11, B). The focus was on the electrode D, n°3 which was the third deepest contact on this electrode. During the sessions of stimulation, the interference was recorded, with dipole and octupole. The dipole's current has been turned on to reach the current recorded during the octopole stimulation session (FIG. 11, C-D). In FIG. 11, E-F, the z score of the spike rate is display. A positive z score demonstrates that mainly on the electrode D, a higher spike rate has been recorded compared to the average per session on all the electrodes. Thus, since the higher spiking rate was on electrode D, it was selected among the other this one to correlate electrophysiological data and envelope of stimulation.

Indeed, the amplitude of the envelope of all the session across all the electrode has been calculated. During the dipole, there is a higher spike rate detected on the shallow part of the electrode D (spearman correlation r=−0.64, p=0.009).

Finally, when adding the different pairs of stimulation in order to create an octopole, it has been shown that the spiking rate evolve positively with the augmentation of the amplitude of the envelope. Indeed, these results tends to show that with a higher focality, spiking generation is correlated with the amplitude of the envelope of stimulation (spearman correlation, r=0.55, p=0.034)

Taken together these results on a deep anesthetize monkey demonstrated that the stimulation with TI has an impact on the intra cranial electrophysiology. Indeed, a focal stimulation with multipolar TI at depth with a 50 Hz envelope evoked spikes positively correlated with the amplitude of the envelope of stimulation. Following this, it was decided to perform multipolar TI stimulation on an awake monkey to demonstrate that it can target a specific deep region and to evoked activity in a non-invasive way.

### Activity Evoked by Multipolar Temporal Interference Stimulation in an Awake Monkey

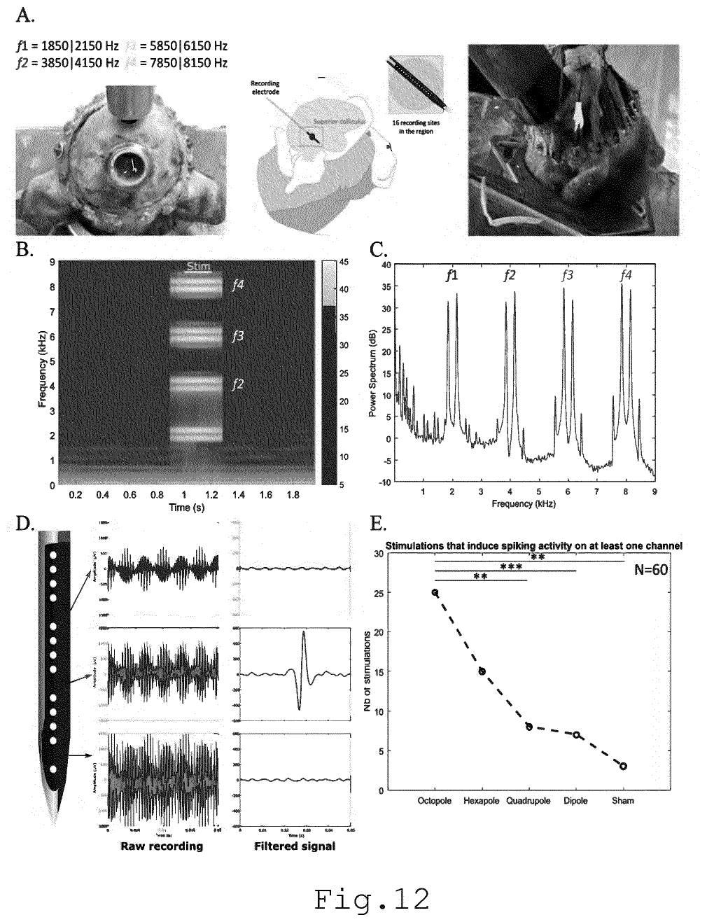

To further analyze the possibility to induce an electrophysiological response, an experiment was carried out on an awake monkey, with an electrode implanted into the superior colliculus (SC). This region is well known to have a high impact on the eye movement encoded by a spiking activity into very specific area of SC. To record the activity in the SC, a 16 channels electrodes was placed and then, the skin electrodes were placed, as shown in FIG. 12, A.

This figure illustrates that Spiking activity has been elicit in an awake monkey. In FIG. 12, A, is represented a picture of the new position of the skin electrodes in order to target the superior colliculus. A 16 channels electrode was placed into the target and gave the possibility to record both electrophysiological signal and artefact of the TI stimulation. In FIG. 12, B, it is shown frequencies used for the stimulation and the corresponding pairs. For this experiment, a 300 Hz gap was used to evoked activity. Pairs with higher frequencies were placed closer to the animal face due to the less sensations on the skin provoked. In FIG. 12, C, it is shown time frequency and periodogram of one of the 200 ms of TI—here only octopole is display—. The different frequencies applied are visible and a lowpass filter <100 Hz is enough to get rid of the artefact of stimulation on the recorded data.

Thus, a 100 Hz envelope was used to evoked activity in the subcortical structure SC with high frequencies carriers in order to be still able to analyzed the electrophysiological data by applying a 1000 Hz lowpass filter (FIG. 13, B, C). Indeed, the spiking activity of interest was in between 130 Hz and 300 Hz. After filtering the data, identifying and counting the number of spikes evoked by the various TI stimulation (dipole, quadrupole, hexapole, octopole, Sham) a Khi 2 analysis was performed to assess the impact of the stimulation on the SC (Khi2 Octopole, Quadrupole, Hexapole, Dipole, Sham: p-value=0.0001). On 60 stimulation trials, the octupole stimulation was able to evoked spiking on 25 stimulations within 0.5 s (Khi2 Octopole vs Quadrupole: p-value=, Octopole vs Hexapole: p-value=, Octopole vs Dipole: p-value=, Octopole vs Sham: p-value=). Then, the number of trials having an effect keeps decreasing by having less than ⅙ trials evoking a spike firing for quadrupole and dipole. Finally, Sham stimulation (suppressing the 100 Hz gap in the stimulation frequencies) have a small effect where only 3 trials evoked an activity.

Finally, taken together these results show that it is possible to get a response from a very specific target the superior colliculus by positioning the skin electrodes in the right placed and to stimulate with an enough amount of pairs. Furthermore, it allows to see that the stimulation did not evoked any itching, pain nor blinking during the stimulations sessions.

Thus, it has been demonstrated that it is feasible to create a completely customizable non-invasive deep brain stimulation, where the size of the deep brain target and the intensity of stimulation can be selected independently. Specifically, the superposition of multiple 50 Hz envelopes in a single point was demonstrated to create a single overlapping envelope with an amplitude significantly greater than the individual envelopes. Using different electric field trajectories allowed to increase the focality, while the multiple electric fields intersect at the same point, they share different path because of the different position of their stimulator. This is interesting as it allows a local maximum at the targeted point while reducing the amplitude modulated signal around it. As getting further away from the targeted point, it was noticed that the different 50 Hz envelopes are not in phase anymore, they interact with each other resulting in destructive interference and frequency modifications that cannot induce activity anymore.

Regarding the impact of TI and multipolar TI on the intracranial electrophysiological recordings, it is shown that with multipolar TI is able to evoke spikes and it is positively correlated with the amplitude of the envelope. Interestingly, for sessions of TI with Dipole, it is shown that there is a significant negative correlation, respectively, in between the spiking rate and the amplitude of the envelope. 50 Hz stimulation in epilepsy allow different possibilities depending on the scale of the stimulation. In clinics, 50 Hz stimulation via intracranial SEEG electrodes lead to the generation of spiking activities to define the areas implicated in the epileptogenic zone. It is also used in animal research where it is possible to induced recurrent epileptic seizures with an intracranial electrode placed in the hippocampus. But when it comes to larger areas, it has been shown that 50 Hz stimulation could have opposite effect. Indeed, TransMagneticalStimulation (TMS) repetition of a 50 Hz stimulation changes the EEG in patients with temporal lobe epilepsy. Applied on both side of anterior temporal region of patients, this stimulation can decrease the number of spikes recorded during 10 min. In the end, it appears that multipolar TI stimulation, and moreover octopole, could reproduce the effect of an implantable stimulation whereas classic TI stimulation could involve more than a sub cortical structure.

After assessing the safety of multipolar TI stimulation on a deeply anesthetized monkey, and finding that the stimulation could indeed be safely performed, stimulation could then be performed in an awake animal. The SC is a subcortical structure responsible for the production of visual saccades. Saccades are preceded by spiking activity of visuomotor and motor neurons in the intermediate and deep layers of the SC. The superior colliculus maps saccadic eye movements on a topological map, and thus depending on where the neural activity occurs, different eye movements will be evoked. SC was stimulated with TI and multipolar TI (quadrupole, hexapole, octopole) and found that with more pairs, more spikes were elicited. To easily get rid of the TI and multipolar TI artefacts of stimulation, a simple lowpass filter can be used only if the recording system is not saturated. If it is, —the signal will be clipped, and it is not possible to calculate the amplitude of the envelope of stimulation. —An artefact at the frequency of interest will be included in the raw signal and the lowpass filtering does not work.

To avoid saturating the recording system, the amplitude of stimulation was not increased. It seems that the multipolar TI stimulation was able to evoke a neural response however this response was below the threshold to elicit a behavioral response. Until now, there is no published document that allowed the possibility to stimulate at depth and recording units in SC at the same time, which seems to be achieved with multipolar TI. Also, since the SC encodes the vectors of eye movements on a topological map saccade in the wanted direction might be evoked by only changing the focus of stimulation.

This invention can safely be used in the non-human primate (NHP) without eliciting any adverse reaction to neither sham nor TI/multipolar TI stimulations. Moreover, due to the central position of the SC within the brain, skin electrodes were placed close to the eyes. Since it has been showed that the higher the frequencies are, the less sensation is evoked, the pairs with higher frequencies were placed closer to the NHP's eye. Thus, although these electrodes were close to the eyes, stimulation did not evoke blinking at any point of the stimulation.

The current trajectory of these electric fields depends on only a few parameters: the current amplitude, the permittivity and conductivity values of the medium and the position and geometry of the electrodes that specifically deliver current. Bearing this in mind, it is possible to influence the trajectory of the electric fields to efficiently reach the targeted zone. The further away the electrodes of a stimulating pair are, the stimulator and its bound electrode, the deeper the current tend to penetrate. This works intuitively on simple shape, but for more elaborate structure we need to consider the substructures and their different physical properties.

Finally, temporal interference stimulation is a unique method of non-invasive brain stimulation (DBS) using transcutaneous electrodes which allows the targeting and stimulation of deeper brain structures without unwanted stimulation of shallower cortical structures. The DBS property of TI has been previously demonstrated, however, the problem of decoupling stimulation focality from stimulation intensity has not been addressed. In this invention, the problem is directly solved with implementing multipolar TI (mTI) stimulation which allows control over the size of the region stimulated and the intensity of stimulation in the region. The multipolar TI according to the invention instead uses multiple carrier frequencies to create multiple overlapping envelopes allowing the size of the stimulated region to be modified independently of the intensity of the stimulation. This description presents both a theoretical explanation of the concept of mTI along with experimental data gathered from Rhesus Monkeys comparing our technique's focality to the focality of the standard temporal interference stimulation technique. It is shown that it is possible to increase the focality and to still be able to evoke electrophysiological activity at depth in two different regions of the brain in anesthetize and awake monkey.

1. A deep brain stimulation system comprising:
at least four stimulation pairs of electrodes,
each of the stimulation pairs of electrodes providing an electric stimulation at a respective carrier frequency,
wherein
a mean value between the carrier frequency of any first stimulation pair of electrodes and the carrier frequency of any second stimulation pair of electrodes defines a first mean carrier frequency,
a mean value between the carrier frequency of any third stimulation pair of electrodes and the carrier frequency of any fourth stimulation pair of electrodes defines a second mean carrier frequency,
a difference between the first and second mean carrier frequencies is equal to or greater than 200 Hz.

2. The system of claim 1, wherein the system comprises from four to a number (n) higher than four of stimulation pairs of electrodes,
and wherein
a difference between the carrier frequency of any first stimulation pair of electrodes and the carrier frequency of any second stimulation pair of electrodes defines a first envelope frequency,
a difference between the carrier frequency of any third stimulation pair of electrodes and the carrier frequency of any fourth stimulation pair of electrodes defines a second envelope frequency,
a difference between the carrier frequency of any subsequent pth stimulation pair of electrodes, 4<p<n, and the carrier frequency of another subsequent qth stimulation pair of electrodes, g=p+1, defines a subsequent rth envelope frequency, r=q/2, and
values of the first, second, and any subsequent rth envelope frequencies are less than or equal to 500 Hz.

3. The system of claim 1, wherein the carrier frequencies of the stimulation pairs of electrodes are greater than 1000 Hz.

4. The system of claim 1, wherein a difference between any two mean carrier frequencies is equal to or greater than 800 Hz.

5. The system of claim 1, wherein the carrier frequency of the first stimulation pair of electrodes is 1250 Hz, the carrier frequency of the second stimulation pair of electrodes is 1300 Hz, the carrier frequency of a third stimulation pair of electrodes is 2150 Hz, and the carrier frequency of a fourth stimulation pair of electrodes is 2200 Hz.

6. The system of claim 2, configured so that the phase of the envelopes is controlled.

7. The system of claim 1, wherein a stimulation amplitude of the electrodes is in a range of from 10 μA to 2500 μA.

8. The system of claim 1, wherein a current is applied to the stimulation pairs of electrodes.

9. The system of claim 1, further comprising one or more current sources, the one or more current sources being electrically connected to the pairs of electrodes.

10. A method of performing deep brain stimulation in a patient, comprising:
providing the system according to claim 1, and
applying the electrical stimulations of the at least four pairs of electrodes to the patient.

11. The method of claim 10, wherein a phase of the envelopes is controlled.

12. The method of claim 10, wherein a stimulation amplitude of the electrodes is increased in steps in a range of from 30 μA to 100 μA.

13. The method of claim 10, wherein the stimulation is biphasic, with bipolar pulses.

14. The method of claim 10, wherein coordinates of the electrodes on the scalp are calculated based at least on a predefined simulation for the stimulation.

15. The method of claim 10, comprising:
providing the at least four stimulation pairs of electrodes, each of the stimulation pairs of electrodes providing an electric stimulation at a respective carrier frequency,
allowing a mean value between the carrier frequency of any first stimulation pair of electrodes and the carrier frequency of any second stimulation pair of electrodes to define a first mean carrier frequency, and a mean value between the carrier frequency of any third stimulation pair of electrodes and the carrier frequency of any fourth stimulation pair of electrodes to define a second mean carrier frequency,
wherein a difference between the first and second mean carrier frequencies is equal to or greater than 200 Hz.

16. The method of claim 11, wherein the phase of the envelopes is controlled by using a predefined phase modulation.

17. The method of claim 12, wherein a stimulation amplitude of the electrodes is increased in steps of 50 μA.

18. The method of claim 14, wherein the coordinates of the electrodes on the scalp are calculated using a finite element method.

19. The system of claim 2, wherein the values of the first, second, and any subsequent nth envelope frequencies are in a range of from 180 Hz to 1 Hz.

20. The system of claim 6, configured so that the phase of the envelopes is controlled by using a predefined phase modulation.
